(19) United States
(12) Patent Application Publication
(10) Pub. No.: US 2016/0375213 A1
(43) Pub. Date: Dec. 29, 2016

(54) Pursed Lip Breathing Apparatus

(71) Applicant: George M. Zlupko, Altoona, PA (US)

(72) Inventor: George M. Zlupko, Altoona, PA (US)

(21) Appl. No.: 15/197,232

(22) Filed: Jun. 29, 2016

Related U.S. Application Data
(60) Provisional application No. 62/185,787, filed on Jun. 29, 2015.

(51) Int. Cl. A61M 16/04; A63B 23/18

(52) U.S. Cl. CPC A61M 16/0488; A63B 23/18

(57) ABSTRACT

Provided is a pursed lip breathing apparatus and method of use thereof. The apparatus includes an elongated member having a first end, a second end, and a central cavity extending between the first end and the second end. A first opening of a first size is located at the first end and a second opening of a second size is located at the second end. The first opening accommodates a user's lips and is larger than the second opening. The central cavity includes a first end portion located in communication with the first opening, a second end portion in communication with the second opening, and a tapered portion between the first and second end portions. The method includes bringing the apparatus to one's mouth, placing one's lips against or around the first end, and breathing out of one's mouth into the first opening.

### CROSS REFERENCE TO RELATED APPLICATION

This application claims priority to U.S. Provisional Patent Application No. 62/185,787, filed on Jun. 29, 2015, the entire contents of which are hereby incorporated by reference.

### BACKGROUND

1. Field of the Invention

This invention relates to a breathing apparatus used for respiration therapy, and particularly to a pursed lip breathing apparatus that can assist the user to control shortness of breath and other breathing-related afflictions.

2. Description of Related Art

Pursed lip breathing is a breathing technique that typically involves exhaling through pursed lips and inhaling through the nose with the mouth closed. Pursed lip breathing has been shown to provide various therapeutic effects, including relieving shortness of breath. Devices to assist users in engaging in pursed lip breathing exercises are known. However, these devices are typically not sufficiently easy to use, are expensive, and are overly and unnecessarily complex for most applications. For example, United States Patent Application Publication No. 2007/0089740 to Baumert et al., which is expressly incorporated herein by reference, discusses a pursed lip breathing apparatus that includes a housing defining an air channel through which a user breathes as well as a feedback system to provide the user with information regarding his or her breathing to motivate and encourage the user during breathing exercises and to monitor the therapeutic effect of his or her breathing.

### SUMMARY

Provided is a pursed lip breathing apparatus and method of use thereof. The apparatus includes an elongated member having a first end, a second end, and a central cavity extending between the first end and the second end. A first opening of a first size is located at the first end and a second opening of a second size is located at the second end. The first opening accommodates a user's mouth and is larger than the second opening. The central cavity includes a first end portion located in communication with the first opening, a second end portion in communication with the second opening, and a tapered portion between the first and second end portions. In one non-limiting embodiment, the first end portion transitions to the tapered portion which transitions to the second end portion. In this embodiment, the first end portion of the central cavity is contiguous with the tapered portion, and the tapered portion is contiguous with the second end portion.

In some non-limiting embodiments, the first opening is between ⅜″ and ⅞″ in diameter, such as between ½″ and ¾″ in diameter.

In some non-limiting embodiments, the second opening is between ⅛″ and 5/16″ in diameter, such as between 3/16″ and ¼″ in diameter.

In some non-limiting embodiments, the tapered portion has a length of between 1.5″ and 3″.

In one non-limiting embodiment, the apparatus includes a collar disposed near the first end portion. The collar can include a collar opening that can accommodate a lanyard, string, cable, or other similar object that can be used to hold or store the apparatus while it is not in use.

Also provided is a method of using the apparatus described above. The method includes bringing the apparatus to one's mouth, placing one's lips against or around the first end, and breathing out of one's mouth into the first opening. The method may also include inhaling through the nose and repeating the breathing exercise while maintaining the position of one's lips against or around the first end.

### DESCRIPTION OF THE DRAWINGS

FIG. 1 is an elevational view of one embodiment of a pursed lip breathing apparatus according to the present invention;

FIG. 2 is a plan view of the first end of the apparatus of FIG. 1;

FIG. 3 is a plan view of the second end of the apparatus of FIG. 1; and

FIG. 4 is a cross section of the apparatus of FIG. 1 taken along line A-A.

### DETAILED DESCRIPTION OF THE PREFERRED EMBODIMENTS

As used herein, all numbers expressing dimensions, physical characteristics, percentages, and the like, are to be understood as being modified in all instances by the term “about” unless otherwise noted.

The invention is directed to a pursed lip breathing apparatus and method of use thereof.

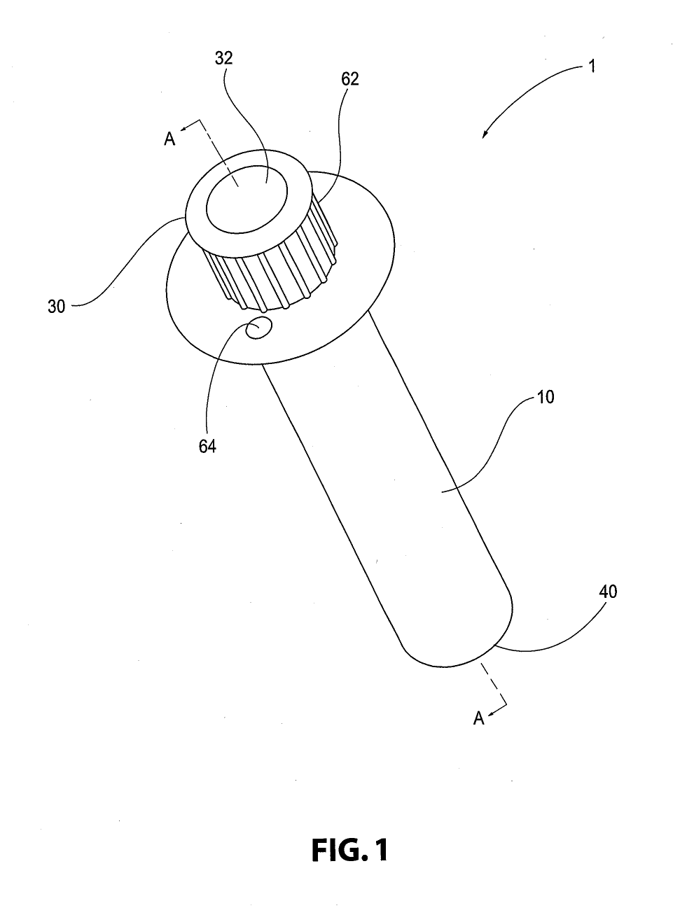

With reference to FIGS. 1-4, the apparatus 1 includes an elongated member 10 extending between a first end 30 and a second end 40. The elongated member 10 is preferably cylindrical, though other cross-sectional shapes, such as octagonal, hexagonal, square, oval, etc. are possible. The apparatus 1 is preferably made of plastic, such as a polyurethane, polyurethane, polystyrene, PVC, or polypropylene, though other plastics can be used as well. Preferably, the material used to construct apparatus 1 is a cost-effective moldable material, like plastic, that is durable enough to be subject to sterilization and washing without becoming deformed in any appreciable way. The material may also include, either as a surface coating or embedded therein, antimicrobial compounds to resist the growth of microbes following use of the apparatus. At least the outer surface of apparatus 1 may be made from, or coated with, a material that is susceptible to being printed upon to allow, for example, logos and advertisements to be printed on the exterior surface thereof. Alternatively, or additionally, apparatus 1 may include an adhesive sticker affixed to the outer surface, where said sticker includes a logo or advertisement.

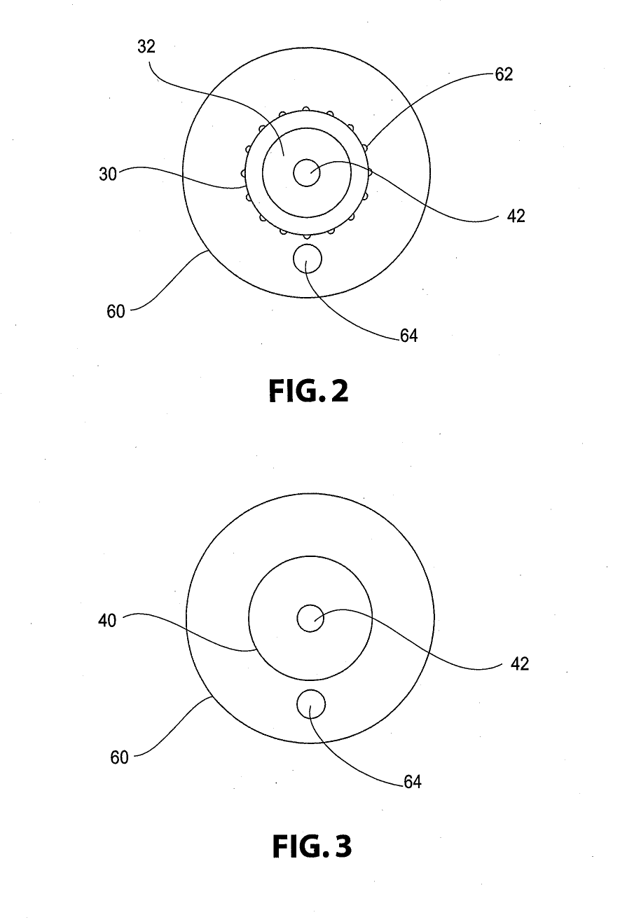

The first end 30 includes a first opening 32 that is of a first size. The first size is selected so as to accommodate a person's lips while those lips are pressed against or placed around the first end 30. A preferred dimension of the first opening 32 can be between about ⅜″ and about ⅞″ in diameter, such as between about ½″ and about ¾″ in diameter, or about ⅝″ in diameter, though this dimension may vary by, for example, an additional 15% in either direction (i.e., smaller or larger). A first opening 32 of this size is useful for an apparatus 1 intended for use by an average-sized adult. Apparatus 1 may also be dimensioned so as to accommodate a smaller sized adult or child, in which case the dimensions may be reduced by, for example, 5-15%. The apparatus 1 may also be custom fitted for a particular user based on that user's preferences and mouth size. The overall length of elongate member 10 may be between about 3″ and about 7″, such as between about 4″ and about 6″, or about 5″, though this amount may be increased by 15% or more. As shown in FIGS. 1 and 2, first end 30 may have a set of evenly spaced ribs 62 located around it.

The second end 40 is disposed on an opposite end of elongate member 10 from first end 30. Second end 40 has a second opening 42 that can be axially disposed at the center of second end 40, as depicted in the figures. Second opening 42 is of a size which is generally smaller than the size of first opening 32 so as to restrict airflow through the elongated member 10 to some extent. In some non-limiting embodiments, second opening 42 is between about ⅛″ and about 5/16″ in diameter, such as between about 3/16″ and about ¼″ in diameter, or about 3/16″ in diameter. However, as explained below, airflow restriction can be accomplished through the shape of the central cavity 20, allowing for second opening 42 to be of a larger size without departing from the scope of the invention. Second opening 42 serves as an outlet for air that enters the central cavity 20 from first opening 32, such as when a user exhales through first opening 32.

As mentioned, apparatus 1 includes a central cavity 20. Central cavity 20 extends through elongated member 10, and typically through the axial center of elongated member 10. Central cavity 20 extends from first end 30 to second end 40 and serves as a conduit connecting first opening 32 with second opening 42. In a preferred embodiment, central cavity 20 includes a first end portion 22 disposed at the first end 30, a tapered portion 24 disposed adjacent to (e.g., contiguously with) first portion 22, and a second end portion 26 disposed adjacent to (e.g., contiguously with) both tapered portion 24 and second end 40. First end portion 22 can have a diameter equivalent to, or nearly equivalent to, that of first opening 32 and a length of between about 0.5″ and 1.0″, such as about 0.75″. Tapered portion 24 has a tapered surface 50 which can taper the diameter of central cavity 20 from that of first end portion 22 to that of second end portion 26. In doing so, tapered portion 24 and tapered surface 50 provide resistance to airflow through the central cavity 20. In certain non-limiting embodiments, tapered portion 24 can have a length of between about 1.5″ and 3″, such as about 2″. Second end portion 26 can be of a constant diameter along its length, and typically this diameter is that of second opening 42, which may also coincide with the final diameter of tapered portion 24. These dimensions can be adjusted by about 15% in either direction, in a manner similar to described above. In addition, central cavity 20 can include other portions as well, such as multiple tapered portions separated by a length of constant diameter. Central cavity 20 may also include less portions, such as by terminating the tapered portion 24 at the second end 40, thereby eliminating the second end portion 26, by beginning the tapered portion 24 at the first end 30, thereby eliminating the first end portion 22, or both. The inner surface of central cavity 20 can be coated with, or embedded with, an antimicrobial substance to lessen the risk that unwanted microbes grow on this surface. The air passage formed by central cavity 20, including, tapered portion 24 and tapered surface 50, help simulate and replicate the action of breathing through lips that are “pursed” or “puckered,” but in a more controlled and reproducible manner.

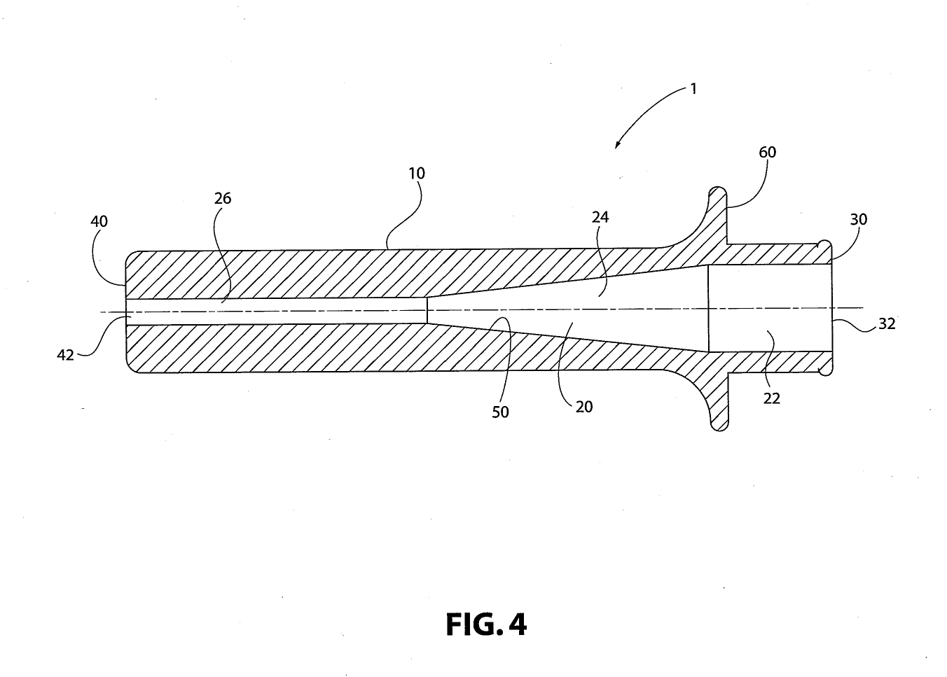

Apparatus 1 can also include a collar 60. Collar is typically positioned near first end 30. Collar 60 serves to assist a user in holding apparatus 1 and creating a seal around the lips when positioning the lips around the first end 30. The dimensions of collar 60 can be determined based on, for example, user and/or manufacturing preference. In certain non-limiting embodiments, collar 60 is positioned at or near the point where the tapered portion 24 begins, as shown in FIG. 4. Collar may be about ⅛″ thick, such as between about 1/16″ and about 3/16″ thick, and have a diameter that is approximately 2-3 times larger than the diameter of the first opening 32. Extending from collar around first end 30 are ribs 62. These ribs 62 can assist a user in positioning his or her lips around the first end 30 during use and provide added comfort. Collar 60 can include a collar opening 64. Collar opening 64 is designed to accommodate a lanyard, string, rope, cable, or other object which can extend from apparatus 1 and which may assist the user in holding or storing apparatus 1 while apparatus 1 is not in use. For example, collar opening 64 may accept a metal ring, which itself is attached to a fabric lanyard. The lanyard can then be worn by the user around his or her neck to enable easy access to apparatus 1 and help prevent misplacement thereof. Lanyard can also be used to store apparatus 1 on a hook or bed post when apparatus 1 is not in use. Lanyards are generally known in the art. Logos or advertisements can be printed on the lanyard used with apparatus 1 to offer another marketing opportunity.

Apparatus 1 can be used in any circumstance where pursed lip breathing may assist the user. This includes, but is not limited to, circumstances where the user wishes to improve ventilation, release trapped air in the lungs, keep his or her airways open longer so as to decrease the work of breathing, slow the breathing rate, improve breathing patterns, relieve shortness of breath, and/or cause general relaxation. To use apparatus 1, user brings apparatus 1 to his or her mouth so as to place his or her lips either against or around first end 30. The user then exhales slowly through first opening 32 so that air passes through central cavity 20 and out second opening 42. User can then inhale, either through the nose with the apparatus 1 still in place or by removing apparatus from his or her mouth and inhaling through the mouth. Preferably, user maintains apparatus 1 positioned at or near the mouth and inhales through the nose. The user can then repeat the exhale process described above. Apparatus 1 assists the user by duplicating the effect of proper pursed lip breathing in a controlled and easily reproducible way.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements. For example, it is to be understood that the present invention contemplates that, to the extent possible, one or more features of any embodiment can be combined with one or more features of any other embodiment.

What is claimed is:

1. A pursed lip breathing apparatus, comprising:
an elongated member having a first end, a second end, and a central cavity extending between the first end and the second end;
a first opening of a first size located at the first end; and
a second opening of a second size located at the second end, wherein the second size is smaller than the first size, and
wherein the central cavity includes a first end portion in communication with the first opening, a second end portion in communication with the second opening, and a tapered portion between the first and second end portions.

2. The apparatus of claim 1, wherein the first size is between ⅜″ and ⅞″ in diameter.

3. The apparatus of claim 2, wherein the first size is between ½″ and ¾″ in diameter.

4. The apparatus of claim 1, wherein the second size is between ⅛″ and 5/16″ in diameter.

5. The apparatus of claim 4, wherein the second size is between 3/16″ and ¼″ in diameter.

6. The apparatus of claim 1, wherein the tapered portion has a length of between 1.5″ and 3″.

7. The apparatus of claim 1, wherein the first end portion is contiguous with the first opening, the second end portion is contiguous with the second opening, and the tapered portion is contiguous with both the first end portion and the second end portion.

8. The apparatus of claim 1, further comprising a collar disposed near the first end portion, wherein the collar includes a collar opening.

9. A pursed lip breathing apparatus, comprising:
an elongated member having a first end, a second end, and a central cavity extending between the first end and the second end;
a first opening of between ½″ and ¾″ in diameter located at the first end; and
a second opening of between 3/16″ and ¼″ in diameter located at the second end,
wherein the central cavity includes a first end portion contiguous with the first opening, a second end portion contiguous with the second opening, and a tapered portion contiguous with both the first end portion and the second end portion.

10. A method of pursed lip breathing, comprising:
bringing a pursed lip breathing apparatus to a mouth so as to position a portion of the mouth against or around the first end; and
exhaling a first breath of air into the first opening,
wherein the pursed lip breathing apparatus comprises:
an elongated member having a first end, a second end, and a central cavity extending between the first end and the second end;
a first opening of a first size located at the first end; and
a second opening of a second size located at the second end, wherein the second size is smaller than the first size, and
wherein the central cavity includes a first end portion in communication with the first opening, a second end portion in communication with the second opening, and a tapered portion between the first and second end portions.

11. The method of claim 10, wherein the first size is between ⅜″ and ⅞″ in diameter.

12. The method of claim 11, wherein the first size is between ½″ and ¾″ in diameter.

13. The method of claim 10, wherein the second size is between ⅛″ and 5/16″ in diameter.

14. The method of claim 13, wherein the second size is between 3/16″ and ¼″ in diameter.

15. The method of claim 10, wherein the tapered portion has a length of between 1.5″ and 3″.

16. The method of claim 10, wherein the first end portion is contiguous with the first opening, the second end portion is contiguous with the second opening, and the tapered portion is contiguous with both the first end portion and the second end portion.

17. The method of claim 10, wherein the first size is between ½″ and ¾″ in diameter, the second size is between 3/16″ and ¼″ in diameter, the tapered portion has a length of between 1.5″ and 3″, the first end portion is contiguous with the first opening, the second end portion is contiguous with the second opening, and the tapered portion is contiguous with both the first end portion and the second end portion.

18. The method of claim 10, further comprising:
inhaling through a nose while the portion of the mouth remains positioned against or around the first end; and
exhaling a second breath of air into the first opening.

19. The method of claim 18, further comprising repeating the inhaling and exhaling steps at least five times.
